(19) United States
(12) Patent Application Publication
(10) Pub. No.: US 2005/0260253 A1
(43) Pub. Date: Nov. 24, 2005

(54) Transmucosal gel formulations

(75) Inventors: Amedeo Leonardi, Milan (IT); Lino Pontello, Milan (IT); Fabio Berlati, Rho (IT)

Correspondence Address: DARBY & DARBY P.C., P. O. BOX 5257, NEW YORK, NY, 10150-5257, US

(73) Assignee: Recordati Ireland Limited, Ringaskiddy (IE)

(21) Appl. No.: 11/134,743

(22) Filed: May 20, 2005

Related U.S. Application Data
(60) Provisional application No. 60/573,241, filed on May 20, 2004.

(51) Int. Cl. A61K 31/517

(52) U.S. Cl. 424/448; 514/252.17

(57) ABSTRACT

This invention relates to pharmaceutical formulations for the transmucosal delivery of 4-amino-6,7-dimethoxy-2-{4-[(2-isopropyl-6-methoxyphenoxy)acetyl]-1-piperazinyl}quinazoline (Rec 15/2615), which has the formula: 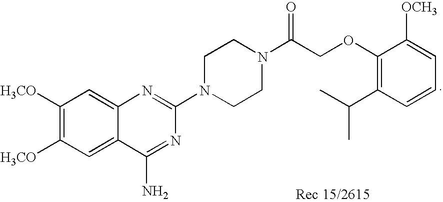 The formulations are useful in methods for enhancing the sexual act for females, including treatment of female sexual dysfunction.

### CROSS-REFERENCE TO RELATED APPLICATION

The present application claims priority under 35 U.S.C. §119(e) of provisional application 60/573,241, filed May 20, 2004, which is hereby incorporated herein by reference in its entirety.

### FIELD OF THE INVENTION

This invention relates to pharmaceutical formulations for the transmucosal delivery of 4-amino-6,7-dimethoxy-2-{4-[(2-isopropyl-6-methoxyphenoxy)acetyl]-1-piperazinyl}quinazoline (Rec 15/2615). The formulations are useful in methods for enhancing the sexual act for females, including treatment of female sexual dysfunction.

### BACKGROUND OF THE INVENTION

Sexual response in women is generally classified into four stages: excitement, plateau, orgasm, and resolution. Masters and Johnson, Human Sexual Response (Boston, Mass.: Little, Brown & Co., 1966). Sexual excitement is initiated by any of a number of psychogenic or somatogenic stimuli and must be reinforced to result in orgasm. With continued stimulation, excitement progresses in intensity into a plateau stage, from which the individual can shift into orgasm. The orgasmic stage is characterized by a rapid release from the physiological effects associated with arousal, vasoconstriction and muscular tension. Estrogens magnify the sexual responses; however, sexual responses may also occur in estrogen-deficient individuals.

During the various stages of the female sexual response, characteristic genital and extragenital responses occur. The female sexual response initiates with a stimulation which causes vasocongestion and swelling, and results in lubrication of the vagina, in preparation for penis insertion. Vasocongestion and muscular tension increase progressively, primarily in genital tissue, and are manifested by increased blood flow and elevated luminal oxygen tension. Lubrication is due to formation of an exudate that saturates the fluid reabsorptive capacity of the vaginal epithelium and which, together with genital congestion, produces the so-called orgasmic platform which is a prelude to orgasm.

Female sexual dysfunction (FSD) describes women who are indifferent or hostile to sexual intercourse, who have no response to sexual advances or stimulation, or who are unable to achieve orgasm during sexual intercourse. FSD is generally recognized to consist of four components: decreased sexual desire; decreased sexual arousal; dyspareunia; and persistent difficulty or inability to achieve orgasm. It has been estimated that 43% of women in the United States have experienced FSD (Laumann, E., et al., Sexual Dysfunction in the United States Prevalence and Predictors. JAMA, 1999, 281: 537-544). Symptoms of FSD include, without limitation, inability to attain or maintain sexual excitement, lack of genital lubrication or swelling, reduced vaginal lubrication, reduced clitoral or labial engorgement, reduced clitoral or labial sensation, and reduced or lacking vaginal smooth muscle relaxation. Clinical manifestations of FSD include, but are not limited to, hypoactive sexual desire disorder, sexual aversion disorder, sexual arousal disorder, orgasmic disorder, and sexual pain disorders (e.g., dyspareunia and vaginismus). Female sexual arousal disorder (FSAD) is the persistent or recurrent inability to attain, or to maintain, sufficient sexual excitement, which causes personal distress. It may be expressed as lack of subjective excitement, lack of genital response, such as lubrication and swelling, or lack of other somatic responses. FSAD is one form of female sexual dysfunction, and is associated with the excitement phase.

Causes of FSD include, but are not limited to, physical, psychological, or emotional factors, hormone deficiencies, medical or surgical interventions, disruption of the female hormonal system caused by, e.g., natural menopause, surgically or medically induced menopause, or endocrine disorders. For example, a variety of diseases affecting neurological function, including diabetes mellitus and multiple sclerosis, may interfere with or block sexual arousal. More commonly, local pelvic disorders, such as endometriosis and vaginitis, both of which cause dyspareunia (difficult or painful coitus) may also affect a woman's sexual response. In addition, estrogen deficiency, causing vaginal atrophy and dyspareunia, is a common cause of sexual dysfunction. For a discussion of other causes of female sexual dysfunction, see, e.g., Kaplan, The Evaluation of Sexual Disorders: Psychological and Medical Aspects (New York: Brunner-Mazel, 1983), and Kolodny et al., Textbook of Sexual Medicine (Boston, Mass.: Little, Brown & Co., 1979).

With respect to particular manifestations of FSD, excitement stage dysfunction generally involves touch sensation impairment, loss of clitoral sensation, vaginal dryness and urinary incontinence. Such excitement phase dysfunction generally results in dyspareunia. Dyspareunia is thought to affect approximately 40% of women, due in large part to inadequate lubrication. Several reasons including stress, anxiety, depression, fatigue, interpersonal conflicts between the partners or more simply ageing, can lead to failure of the vasocongestive response, thereby inhibiting normal vaginal lubrication. Women in this condition may be incapable of achieving a normal sexual response without appropriate treatments (Harrison's Principles of Internal Medicine, 14th Ed., page 291, McGraw-Hill (1998)). It has been estimated that over 40 million women will suffer dyspareunia at some time in their lives. On the order of twenty-five million will experience dyspareunia in the peri- and postmenopausal period (see Kelly, S., 1992, Clinical Practice and Sexuality 8:2 and Sato et al., 1992, Clinical Practices in Sexuality 8:1). Symptomatic treatments generally involve the use of physiologically safe lubricants such as egg white, K-Y surgical lubrication jelly (hydroxyethyl-cellulose), Astroglide®, and Replens®. See, for example, Semmens, 1974, Medical Aspects of Human Sexuality 8:85-86, and Frishmen et al., 1992, Fertility and Sterility 58:630. When symptomatic treatment fails, pharmacological treatment may be indicated.

It has been confirmed that both vaginal vasocongestion and clitoral erection depend on increased blood flow (Park, K., et al., 1997, Int. J. Impot. Res. 9:27-37 (1997)). Moreover, similar to what has been reported for the male sexual organ, the same authors demonstrated that a local injection in the vagina of α1 adrenergic antagonists such as phentolamine can increase blood flow and intravaginal pressure up to levels comparable with those achieved by stimulation of the pelvic nerve. These data clearly indicate that noradrenaline plays an important role in maintaining flaccidity of the organ concerned in the female sexual tract too.

U.S. Pat. No. 6,303,606 describes the use of selective antagonists of the α_{1B}-adrenergic receptor for improvement of sexual dysfunction in both males and females. The compounds disclosed therein can facilitate sexual intercourse, while avoiding the excessive side effects due to acute hypotension. One of the compounds disclosed in the '606 patent is 4-amino-6,7-dimethoxy-2-[4-[(2-isopropyl-6-methoxyphenoxy)acetyl]-1-piperazinyl]quinazoline (Rec 15/2615).

There is an ongoing need for methods and compositions for enhancing female sexual responsiveness and/or treating of FSD, including FSAD. Desirable methods and compositions should be safe, effective, simple to use, and free of undesirable side effects. Accordingly, the present inventors have discovered hydrogel formulations that are effective for the transmucosal administration of Rec 15/2615. The formulations are easy to formulate, simple to apply, have desirable tactile properties, and lead to rapid and efficient point-of-use absorption of Rec 15/2615 when applied to the vaginal mucosa. The formulations are well-tolerated and their use reduces or avoids undesirable local or systemic effects, contributing to good patient compliance. The small number of components in formulations of the invention reduces the risk of irritation or sensitization. The formulations are thus useful in the treatment of FSD, particularly by transmucosal delivery.

### SUMMARY OF THE INVENTION

The invention provides hydrogel formulations effective for the transmucosal delivery of Rec 15/2615 following topical administration to the female genital mucosa.

Accordingly, in certain embodiments, the invention provides a transmucosal delivery preparation comprising a therapeutically effective amount of Rec 15/2615, or a pharmaceutically acceptable salt thereof, and a solubilizing agent, penetration enhancer, and thickening agent, and optionally a humectant, wherein said preparation is adapted to deliver a therapeutic amount of Rec 15/2615 across a female genital mucosal membrane.

In certain embodiments, the invention provides a transmucosal delivery preparation comprising a therapeutically effective amount of Rec 15/2615, or a pharmaceutically acceptable salt thereof, and a solubilizing agent, penetration enhancer, and a thickening/emulsifying agent, and optionally a humectant, wherein said preparation is adapted to deliver a therapeutic amount of Rec 15/2615 across a female genital mucosal membrane.

In certain embodiments, the invention provides a method for treating female sexual dysfunction in a patient in need of such treatment, comprising transmucosally administering the aforementioned transmucosal delivery preparations comprising a therapeutically effective amount of Rec 15/2615 or pharmaceutically acceptable salt thereof to the genital mucosa of said patient in need, to treat said female sexual dysfunction.

In certain embodiments, the invention provides a method of treating female sexual dysfunction in a patient in need thereof, comprising topically administering the aforementioned transmucosal delivery preparations comprising a therapeutically effective amount of Rec 15/2615 or pharmaceutically acceptable salt thereof to the genital mucosal membrane of said patient, thereby treating said female sexual dysfunction.

In certain embodiments, the invention provides a method for enhancing female sexual responsiveness comprising topically administering the aforementioned transmucosal delivery preparations comprising a therapeutically effective amount of Rec 15/2615 or pharmaceutically acceptable salt thereof to the genital mucosal membrane of a female individual, the composition comprising an amount of Rec 15/2615 or pharmaceutically acceptable salt thereof sufficient to increase the blood flow in the clitoris or vaginal wall of said female individual, thereby enhancing the sexual responsiveness of said female individual.

In certain embodiments, the invention provides a method of administering Rec 15/2615 or a pharmaceutically acceptable salt thereof transmucosally to a female patient, which comprises contacting a genital mucous membrane of said patient with the foregoing compositions comprising Rec 15/2615 or pharmaceutically acceptable salt thereof, and maintaining contact of said composition with said genital mucous membrane for a sufficient time period to deliver a therapeutically effective amount of the Rec 15/2615 or pharmaceutically acceptable salt thereof to said patient.

In certain embodiments, the methods described above further comprise combination treatment with other active agents which enhance the sexual act for females, such as, for example and without limitation, prostaglandins, direct vasodilators; and type-V phosphodiesterase inhibitors (PDE-5 inhibitors).

In certain embodiments, the pharmaceutical compositions described above further include other active agents which enhance the sexual act for females, such as, for example and without limitation, one or more of prostaglandins, direct vasodilators; and type-V phosphodiesterase inhibitors (PDE-5 inhibitors).

### BRIEF DESCRIPTION OF DRAWINGS

FIG. 1 is a schematic representation of a tracing obtained in experiments designed to measure the effect of topical administration of Rec 15/2615 on female genital tissue blood flow. S1 and S2 represent the increase in genital tissue oxyhemoglobin induced by pelvic nerve stimulation prior to topical (transmucosal) administration or injection of Rec 15/2615. S3 and S4 represent the increase in genital tissue oxyhemoglobin induced by pelvic nerve stimulation following topical administration or injection of Rec 15/2615 at t=0. The shaded area represents the area-under-the-curve of the increase in genital tissue oxyhemoglobin induced by application of the Rec 15/2615 formulation or drug injection.

FIG. 2 shows the effect of control formulation on genital tissue oxyhemoglobin (OxyHb) concentration. Control formulation without active ingredient was applied at the time indicated by the arrow labeled “gel”. S1-S4 indicate times of pelvic nerve stimulation.

FIG. 3 depicts the genital tissue oxyhemoglobin levels observed following topical vaginal administration of control formulation lacking active ingredient (vehicle) and formulations containing 0.075, 0.15, or 0.3% Rec 15/2615 hydrochloride. Data are shown as mean area-under-the-curve (AUC)+SEM for different considered time intervals.

FIG. 4 shows the change in genital tissue oxyhemoglobin concentration observed following topical vaginal administration of control formulation lacking active ingredient (vehicle) and formulations containing 0.075, 0.15, or 0.3% Rec 15/2615 hydrochloride. Data represent mean±SEM of peaks amplitude obtained upon pelvic nerve stimulation.

FIG. 5A-5D depicts systemic blood pressure observed following topical vaginal administration of control formulation lacking active ingredient (vehicle) (5A) and formulations containing 0.075% (5B), 0.15% (5C), or 0.3% (5D) Rec 15/2615 hydrochloride. Data represent mean±SEM.

FIG. 6 shows the change in genital tissue oxyhemoglobin concentration observed following pelvic nerve stimulation in animals in basal state and 15 and 30 min following vaginal submucosal injection of 100 μg of Rec 15/2615 hydrochloride. Data are mean±SEM. *p=<0.05 versus basal stimulation.

### DETAILED DESCRIPTION OF THE INVENTION

Every patent, patent application and publication, and literature reference mentioned in the present specification is hereby incorporated herein by reference in its entirety.

The transmucosal delivery of Rec 15/2615 or a pharmaceutically acceptable salt thereof can be accomplished by contacting a female genital mucosal membrane, e.g., the vaginal mucosa, with a source of Rec 15/2615 or a pharmaceutically acceptable salt thereof, and maintaining said source in contact with said mucous membrane for a sufficient period for the active ingredient to be transmucosally absorbed and induce the desired therapeutic effect.

The precise amount of active agent administered to achieve the desired therapeutic effect is dependent on numerous factors, such as age and body weight of the individual, condition of the individual, and the desired duration of use. The daily dose of Rec 15/2615 in humans is in the range of about 0.1 mg to about 50 mg, preferably about 0.5 mg to about 10 mg. The plasma concentration in human individuals is about 1 to about 100 ng/mL, preferably about 10 to about 80 ng/mL, and most preferably about 15 to about 70 ng/mL. The variability of therapeutic effect among individuals (interpatient variability) may be diminished by administering the active agent near or directly at the site of action. In a preferred embodiment, active agent is administered topically to female genital tissue, e.g., the vaginal mucosa, clitoris, mons pubis, labia majora, and/or labia minora. A preferred amount of Rec 15/2615 to be administered to an individual is an amount between about 0.5 mg/dose to about 10.0 mg/dose. Formulations comprising Rec 15/2615 are preferably administered topically to female genital tissue from about 5 to about 30 min prior to sexual activity. Most preferably, Rec 15/2615 is administered topically to female genital tissue from about 10 to about 30 min prior to sexual activity. The amount of formulation applied to obtain a particular dose, plasma concentration or therapeutic effect may be varied as needed. For humans, preferably between about 1 ml to about 15 ml and more preferably about 2 to 10 ml of hydrogel is applied with each application. A unit dosage contained in 5 ml of formulation is most preferred.

The formulations of the invention comprise an active agent, and one or more pharmaceutically acceptable excipients. Pharmaceutically acceptable excipients for inclusion in hydrogel formulations include, for example and without limitation, solubilizing, emulsifying, thickening, bioadhesive, and viscosity increasing agents, penetration enhancers, humectants, surfactants, buffering agents, preservative agents, antioxidants, colorants, chelating agents, tonicity agents, flavors, and perfumes. In a preferred embodiment, formulations comprise a solubilizing agent, a penetration enhancer and a thickening agent. Also preferred are formulations consisting of a solubilizing agent, a penetration enhancer and a thickening agent. In another preferred embodiment, formulations comprise a solubilizing agent, a penetration enhancer, a thickening agent and a humectant. In another preferred embodiment, formulations consist of a solubilizing agent, a penetration enhancer, a thickening agent and a humectant. In another preferred embodiment, formulations comprise a solubilizing agent, a penetration enhancer, a thickening agent and an emulsifying agent. In another preferred embodiment, formulations consist of a solubilizing agent, a penetration enhancer, a thickening agent and an emulsifying agent. Formulations may also comprise, for example and without limitation, preservative agents and/or an antioxidants. One of ordinary skill in the art will recognize that a particular agent may have more than one of the aforementioned properties. For example, an agent may have properties that make it effective as both a solubilizing agent and a penetration enhancer. In the Examples set forth below, e.g., diethyleneglycol monoethyl ether (e.g., Transcutol® P), has the properties as being both a solubilizing agent for the active ingredient during preparation of the formulation and as a penetration enhancer in the final formulation, following topical administration to a mucosal membrane. Sepigel™ 305, a mixture of polyacrylamide, C₁₃₋₁₄ isoparaffin and laureth-7, has the properties as being both a thickening agent and an emulsifying agent.

Each of the aforementioned embodiments may further comprise one or more additional active agent. An additional active agent is preferably useful for enhancing female sexual responsiveness and/or treating of FSD.

Hydrogel formulations preferably have the properties of good solubilization of hydrophobic active agents, e.g., without limitation, Rec 15/2615, good penetration into mucosal tissue, e.g., female genital mucosal tissue, leading to effective transmucosal delivery of active agent, pleasant tactile properties, e.g., a non-greasy feel, good adherence to mucosal surfaces, e.g., without limitation, the vaginal mucosal surface, and good tolerability and reduction or elimination of undesirable local effects, which contribute to good patient compliance.

Hydrogel formulations preferably also have a viscosity profile characterized by a higher viscosity at room temperature and a lower viscosity at body temperature, e.g., the temperature of female genital mucosal surfaces. The viscosity at body temperature should preferably be such that the formulation spreads over a mucosal surface, e.g., a female genital mucosal surface, without running. Viscosity values in the range of 10,000-40,000 cPs are preferred.

Hydrogel formulations preferably have pH values between about 4 to about 10. More preferably, hydrogel formulations preferably have pH values between about 4 to about 8 or about 4 to about 7. More preferably, hydrogel formulations have an acidic pH in the range of about 4 to about 6.9. Still more preferably, hydrogel formulations have a pH in the range of about 4.5 to about 6.5. Most preferably, hydrogel formulations have a pH in the range of about 4.8 to about 6.1.

Formulations of the invention can have a clear, colorless appearance or an opaque white or translucent appearance.

In preferred embodiments, hydrogel formulations comprise Rec 15/2615 or a pharmaceutically acceptable salt thereof as active agent. Rec 15/2615 may be synthesized as described in U.S. Pat. No. 6,303,606 (see Example 1). Pharmaceutically acceptable salts include, without limitation, hydrochloride, hydrobromide, mono- or dibasic sulphate, phosphate, mesylate, besylate, tosylate, acetate, propionate, succinate, lactate, malate, maleate, fumarate, benzoate, salycilate, and gentysate salts. Most preferably, the salt is the hydrochloride salt of Rec 15/2615.

Examples of solubilizing agents include, but are not limited to, diethyleneglycol monoethyl ether (e.g., Transcutol® P), alcohols and polyols, such as ethanol, isopropanol, butanol, benzyl alcohol, ethylene glycol, propylene glycol, butanediols and isomers thereof, glycerol, pentaerythritol, sorbitol, mannitol, dimethyl isosorbide, polyethylene glycol, polypropylene glycol, polyvinylalcohol, hydroxypropyl methylcellulose and other cellulose derivatives, cyclodextrins, including cyclodextrin derivatives, e.g., totally or partially alkylated and/or hydroxyalkylyated cyclodextrin ethers, see, e.g., U.S. Pat. No. 6,407,079; ethers of polyethylene glycols having an average molecular weight of about 200 to about 6000, such as tetrahydrofurfuryl alcohol PEG ether (glycofurol, available commercially from BASF under the trade name Tetraglycol) or methoxy PEG (Union Carbide); amides, such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide, and polyvinylpyrrolidone; esters, such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide (Arlasolve DMI (ICI)), N-methylpyrrolidones (Pharmasolve (ISP)), and monooctanoin. Solubilizing agents may be used in combination, for example a cyclodextrin can be used in combination with a mono- or poly-hydroxyalcohol or alcohol ether, such as Transcutol® P.

Examples of penetration enhancers include, but are not limited to, diethyleneglycol monoethyl ether (e.g., Transcutol® P), ethyl alcohol, isopropyl alcohol, lauryl alcohol, salicylic acid, octylphenylpolyethylene glycol, polyethylene glycol 400, propylene glycol, N-decylmethylsulfoxide and DMSO. Penetration enhancers may be used in combination.

A preferred solubilizing agent and penetration enhancer is diethyleneglycol monoethyl ether (e.g., Transcutol® P). Preferred amounts of diethyleneglycol monoethyl ether are from about 1% to about 75%, from about 5% to about 75%, from about 5% to about 50%, from about 10% to about 50%, from about 10% to about 30%, or from about 20% to about 75%, by weight. A more preferred amount of diethyleneglycol monoethyl ether is about 30% to about 49% by weight.

Also preferred as solubilizing agents are cyclodextrins, more preferably β-cyclodextrins. A most preferred cyclodextrin is hydroxypropyl-β-cyclodextrin, available, e.g., as Kleptose® HP. Preferred amounts of cyclodextrin, e.g., hydroxypropyl-β-cyclodextrin, are from about 1% to about 15%, from about 2% to about 10%, from about 3% to about 8%, from about 4% to about 9%, or from about 5% to about 7%. A more highly preferred amount of cyclodextrin, e.g., hydroxypropyl-β-cyclodextrin, is about 5.5% to about 6.5% by weight. A most highly preferred amount of cyclodextrin, e.g., hydroxypropyl-β-cyclodextrin, is about 6% by weight.

Examples of thickening agents include, but are not limited to, hydroxyethyl cellulose (e.g., Natrosol® 250M), methyl cellulose, hydroxypropyl cellulose, hydroxypropyl methyl cellulose, cellulose acetate, ethyl cellulose, methyl hydroxyethyl cellulose, cellulose gum, xantan gum, guar gum, aluminum magnesium silicate and cross-linked acrylic polymers. Preferred thickening agents are those thickening agents that form hydrogels. Thickening agents may also be used in combination.

A preferred thickening agent is hydroxyethyl cellulose (e.g., Natrosol® 250 M). Hydroxyethyl cellulose is typically present in an amount from about 0.5% to about 20% by weight. More preferably hydroxyethyl cellulose is present in an amount from about 0.5% to about 15%, from about 1% to about 15%, from about 1% to about 10%, from about 2% to about 10%, or from about 2% to about 5% by weight. Most preferred are formulations wherein hydroxyethyl cellulose is present in an amount of about 2% by weight.

Examples of humectants include, but are not limited to, propylene glycol, glycerin, sorbitol, urea, 1,3-butylene glycol, hexylene glycol, ethanol, and isopropanol. Humectants may be used in combination.

A preferred humectant is propylene glycol. When included in compositions, propylene glycol is preferably present in an amount from about 0.1% to about 50%, from about 0.5% to about 50%, from about 1% to about 20%, from about 5% to about 20%, or from about 5% to about 15% propylene glycol. Most preferably, when included in compositions, propylene glycol is present in an amount of about 10%.

A preferred thickening/emulsfying agent is Sepigel™ 305 (commercially available from Seppic Corporation, Fairfield, N.J., USA), a mixture of polyacrylamide, C₁₃₋₁₄ isoparaffin and laureth-7. Preferred amounts of Sepigel™ 305 are from about 0.5% to about 20%, from about 1% to about 10%, from about 1% to about 5%, from about 2% to about 5%, or from about 2% to about 4%. A more highly preferred amount of Sepigel™ 305 is about 2% to about 4% by weight. A most highly preferred amount of Sepigel™ 305 is about 3% by weight.

Compositions of the invention may further comprise one or more preservative including, without limitation, antioxidant or antimicrobial agents. Examples of preservatives include, but are not limited to, quaternary ammonium salts, such as quaternium 15, benzalkonium chloride, cetrimide, benzethonium chloride; and imidazolidinyl urea; organic acids, such as sorbic acid, p-hydroxybenzoic acid, and benzoic acid; parabens, such as methyl paraben, ethyl and propyl paraben; alcohols, such as benzyl alcohol and isopropyl alcohol; phenols, such as triclosan, chlorhexidine, and thimerosal; hydantoin derivatives; chloromethylthiazoline; methylisothiazoline; phenoxyethanol; hexetidine; chlorohexydine gluconate. Preferred preservatives include methyl p-hydroxybenzoate and propyl p-hydroxybenzoate. Examples of antioxidants include, but are not limited to, ascorbic acid and its esters, sodium bisulfite, sodium metabisulfite, thiourea, butylated hydroxytoluene, butylated hydroxyanisole, tocopherols, e.g., tocopheryl acetate, tocopheryl palmitate, alkyl gallates, and chelating agents like EDTA and citric acid. When present in a composition of the invention, the amount of a preservative or antioxidant is preferably from about 0.001% to about 1% by weight of the total composition weight, more preferably from about 0.01% to about 0.5% by weight. Preferred antioxidants include tocopheryl acetate and tocopheryl palmitate.

The following are non-limiting embodiments of hydrogel base compositions provided by the present invention:

A composition comprising diethyleneglycol monoethyl ether and hydroxyethylcellulose.

A composition comprising diethyleneglycol monoethyl ether, hydroxyethylcellulose, and propylene glycol.

A composition comprising from about 30% to about 50% diethyleneglycol monoethyl ether, from about 0.5% to about 5% hydroxyethylcellulose, and a non-zero amount, preferably from about 0.1% up to about 20% propylene glycol.

A composition comprising from about 30% to about 50% diethyleneglycol monoethyl ether and from about 1% to about 5% hydroxyethylcellulose.

A composition comprising from about 30% to about 50% diethyleneglycol monoethyl ether, from about 1% to about 5% hydroxyethylcellulose and from about 5% to about 10% propylene glycol.

A composition comprising diethyleneglycol monoethyl ether, hydroxyethylcellulose and cyclodextrin (e.g., hydroxypropyl-β-cyclodextrin).

A composition comprising from about 30% to about 50% diethyleneglycol monoethyl ether, from about 1% to about 5% hydroxyethylcellulose and from about 1% to about 15% cyclodextrin (e.g., hydroxypropyl-β-cyclodextrin).

A composition comprising diethyleneglycol monoethyl ether and Sepigel™ 305.

A composition comprising from about 30% to about 50% diethyleneglycol monoethyl ether and from about 0.5% to about 20% Sepigel™ 305.

The aforementioned compositions may further comprise a therapeutically effective amount of Rec 15/2615. Such preparations are used for the methods of treatment described herein. One of ordinary skill in the art will recognize that the aforementioned compositions may comprise one or more active agents in addition to Rec 15/2615, for use in the methods described herein.

An “effective amount” of a compound for enhancing female sexual responsiveness or treating female sexual dysfunction is an amount that produces a measurable increase in the amount of blood flow in the clitoris or vaginal wall. This measurement can be made by laser Doppler fluorimetry and is well-known to one of ordinary skill in the art. The exact amount to be administered to a patient may vary depending on the state and severity of the disorder and the physical condition of the patient. It will be understood that any significant clinical or statistical improvement is within the scope of this invention. Clinically significant improvement is defined as an improvement perceptible to the patient and/or to the physician.

The pharmaceutical compositions comprising Rec 15/2615 may optionally be used in combination with other active agents which enhance the sexual act for females. Such active agents include, but are not limited to, prostaglandins, for example prostaglandin E₂; direct vasodilators, for example papaverine; and type-V phosphodiesterase inhibitors (PDE-5 inhibitors), for example 1-{[3-(4,7-dihydro-1-methyl-7-oxo-3-propyl-1H-pyrazolo[3,4-d]pyrimidin-5-yl-4-ethoxyphenyl]-4-methylpiperazine, also known as sildenafil, 2-[2-ethoxy-5-(4-ethylpiperazine-1-ylsulphonyl)phenyl]-7-propylimidazo[5,1-f][1,2,4]triazin-4(3H)-one, also known as vardenafil, and (6R,12aR)-6-(1,3-benzodioxol-5-yl)-2-methyl-1,2,3,4,6,7,12,12a-octahydropyrazino[1′,2′:1,6]pyrido[3,4-b]indole-1,4-dione, also known as tadalafil, all commercially available. These compounds may supplement the direct action of Rec 15/2615 in producing the desired effects of enhancing female sexual responsiveness and/or treating FSD.

As used herein, “in combination” means administration of one or more compounds to achieve the desired result of enhancing female sexual responsiveness and/or treating of FSD. Active agents may be used in combination by administering a single dosage form or separate dosage forms. When separate dosage forms are used, active agents used in combination may be administered at the same time or at different times.

The hydrogel formulations comprising Rec 15/2615 may be used to treat FSD. The term “treatment” includes the amelioration of any cause or symptom of FSD, examples of which are set forth above, without limitation.

Formulations may be administered using methods that are well known in the art including, without limitation, via a multi-dose or single-dose tube, syringe or other applicator, such as a monodose plastic ampoule or others.

The methods, tables and examples provided below are intended to more fully describe the preferred embodiments of the invention and to demonstrate its advantages and applicability without limiting its scope.

### EXAMPLE 1

### Rec 15/2615 Hydrogel Formulations

Rec 15/2615 hydrochloride was synthesized according to methods given in U.S. Pat. No. 6,303,606. Preferred formulations include the ingredients and amounts shown in Tables 1-3. Amounts of all ingredients are given as final weight to weight (w/w) (g/100 g).

TABLE 1

| | Batch: | | | | | |
|---|---|---|---|---|---|---|
| | 24/A | 26 | 33-37-52-58 | 36-51 | 50 | 66 |
| REC 15/2615 | 0.5 | 0.5 | 0.3 | 0.15 | 0.075 | 0.3 |
| hydrochloride | | | | | | |
| Diethyleneglycol | 49.0 | 30.0 | 30.0 | 30.0 | 30.0 | 30.0 |
| monoethyl ether | | | | | | |
| (Transcutol ® P) | | | | | | |
| Propylene glycol | — | 10.0 | — | — | — | — |
| Hydroxyethyl | 2.0 | 2.0 | 2.0 | 2.0 | 2.0 | 2.5 |
| cellulose | | | | | | |
| Water | a.s. to 100 | a.s. to 100 | a.s. to 100 | a.s. to | a.s. to 100 | a.s. to |
| | | | | 100 | | 100 |

Manufacturing Method for Compositions of Table 1

Phase 1. Active ingredient Rec 15/2615 hydrochloride was added in portions over a period of 10 min to diethyleneglycol monoethyl ether (Transcutol® P) under gentle stirring. A clear solution was obtained.

Phase 2. Water was heated to 50° C. and added to the Phase 1 solution while mixing. Mixing was continued until a clear solution was obtained. The resulting solution was cooled to room temperature.

Phase 3. The hydroxyethyl cellulose was added in portions over a period of 15 min to the Phase 2 solution under stirring. Stirring was continued to allow the hydroxyethyl cellulose to swell. The resulting hydrogel was subjected to quality control tests (e.g., pH, assay of active ingredient, viscosity).

Phase 4. The hydrogel prepared in Phase 3 was used to fill aluminum tubes or containers adapted for vaginal delivery, e.g., syringes or single-dose vaginal devices.

Typical results were obtained for batch 66: Viscosity 23200 cPs at 23.8° C., pH=5.13, active ingredient assay=101.0% of the nominal amount.

TABLE 2

Batch:

| | P566/98 | P566/101 |
|---|---|---|
| REC 15/2615 hydrochloride | 0.3 | 0.075 |
| Hydroxypropyl-β-cyclodextrin | 6.0 | 6.0 |
| (Kleptose ® HP) | | |
| Diethyleneglycol monoethyl ether | 30.0 | 30.0 |
| (Transcutol ® P) | | |
| Hydroxyethyl cellulose | 2.0 | 2.0 |
| (Natrosol ® 250 M) | | |
| Water | a.s. to 100 | a.s. to 100 |

Manufacturing Method for Compositions of Table 2

Phase 1. The hydroxypropyl-β-cyclodextrin was added to the diethyleneglycol monoethyl ether and water at room temperature while mixing, avoiding formation of agglomerates.

Phase 2. Active ingredient REC 15/2615 hydrochloride was added to the Phase 1 mixture with continued mixing until complete solubilization of active ingredient was obtained.

Phase 3. The hydroxyethyl cellulose was added in portions to the Phase 2 solution under stirring, avoiding the formation of agglomerates. Stirring was continued to allow the hydroxyethyl cellulose to swell. The resulting hydrogel was subjected to quality control tests (e.g., pH, assay of active ingredient, viscosity).

Phase 4. The hydrogel prepared in Phase 3 was used to fill aluminum tubes or containers adapted for vaginal delivery, e.g., syringes or single-dose vaginal devices.

Results: A clear colorless gel was obtained for both compositions. For batch P566/98, the assay of Rec 15/2615 yielded 110% of the nominal amount and a pH of 4.96 (viscosity was not tested). For batch P566/101, the assay of Rec 15/2615 yielded 100% of the nominal amount, a pH of 5.63 and viscosity of 17,100 cPs at 23.8° C.

TABLE 3

Batch:

| | P566/99 | P566/104 |
|---|---|---|
| REC 15/2615 hydrochloride | 0.3 | 0.075 |
| Diethyleneglycol monoethyl ether | 30.0 | 30.0 |
| (Transcutol ® P) | | |
| Mixture of polyacrylamide, C₁₃₋₁₄ | 3.0 | 3.0 |
| isoparaffin and laureth 7 | | |
| (Sepigel ™ 305) | | |
| Water | a.s. to 100 | a.s. to 100 |

Manufacturing Method for Compositions of Table 3

Phase 1. The diethyleneglycol monoethyl ether and water were mixed at room temperature.

Phase 2. Active ingredient REC 15/2615 hydrochloride was added to the Phase 1 mixture with continued mixing until complete solubilization of active ingredient was obtained.

Phase 3. Sepigel™ 305 was added to the Phase 2 solution under stirring, avoiding the formation of agglomerates. Stirring was continued to allow gel formation. The resulting gel having a cream texture was subjected to quality control tests (e.g., pH, assay of active ingredient, viscosity).

Phase 4. The hydrogel prepared in Phase 3 was used to fill aluminum tubes or containers adapted for vaginal delivery, e.g., syringes or single-dose vaginal devices.

Results: An opaque white gel was obtained for both compositions. For batch P566/99, the assay of Rec 15/2615 yielded 106% of the nominal amount and a pH of 5.97 (viscosity of this batch was not tested). For batch P566/104, the assay of Rec 15/2615 yielded 96% of the nominal amount, a pH of 6.04 and viscosity of 34,000 cPs at 23.8° C.

### EXAMPLE 2

### Effects of Topical Vaginal Administration of the Hydrogel Containing Rec 15/2615 on Genital Blood Flow in Female Anesthetized Rabbits

In Vivo Model of Female Sexual Arousal

The effect of Rec 15/2615 formulation on genital blood flow, a physiologic parameters of female sexual arousal, was measured in both the basal, non-aroused state and following stimulation of the pelvic nerve, which mimics the sexually aroused state, in an established in vivo animal model of female sexual arousal (see, e.g., Munarriz, R., et al., 2003, A review of the physiology and pharmacology of peripheral (vaginal and clitoral) female genital arousal in the animal model, J. Urol. 170(2 Pt 2):S40-44; Min, K., et al., 2000, Sildenafil augments pelvic nerve-mediated female genital sexual arousal in the anesthetized rabbit. Intl. J. Impot. Res. 12(Suppl)3:S32-39; Tarcan, T., et al., 2000, Systemic administration of apomorphine improves the hemodynamic mechanism of clitoral and vaginal engorgement in the rabbit. Int J Impot Res. 12:235-240; Park, K., et al., 1997, Vasculogenic female sexual dysfunction: the hemodynamic basis for vaginal engorgement insufficiency and clitoral erectile insufficiency. Intl. J. Impot. Res. 9:27-37). Each of the foregoing references is incorporated herein by reference in its entirety.

Methods

Pelvic Nerve Isolation and Stimulation.

Female New Zealand White rabbits (4.5 to 5 kg) were anesthetized by intramuscular injection of ketamine (35 mg/kg) and xylazine (5 mg/kg) and secured in the supine position. Anesthesia was maintained as needed with additional intramuscular xylazine (5 mg/ml). A midline neck incision (3 cm) was fashioned to access the carotid artery. A 20-gauge angiocatheter was inserted into the carotid artery for systemic blood pressure measurements. Continuous recordings were achieved with a PT300 pressure transducer connected to a PI-1-ACDC signal conditioner module and a Grass 7400 physiological recorder (Grass Instruments Div., Astro-Med, Inc., Warwick, R.I., USA). Body temperature was maintained with an electric heat pad. Following subcutaneous lidocaine injection, the perivesical space was exposed through a mid-line abdominal incision. The pelvic nerve was identified and carefully dissected under the perivesical fat on the postero-lateral aspect of the upper vagina. Bladder contents were aspirated through the bladder wall with an 18 gauge needle and 50 ml syringe. Under direct vision, a bipolar platinum wire electrode was hooked onto the pelvic nerve without cutting the nerve. Unilateral pelvic nerve stimulation was accomplished with a Grass SD9 stimulator set at normal polarity and repeat mode to generate a 30 second train of square waves with 10 V pulse amplitude, 0.8 msec pulse duration and 2-6 Hz frequency. A laser Doppler flowmetry surface probe (model BLF21D, Transonic System Inc, Ithaca, N.Y., USA) was placed into the rabbit vagina and used to confirm unilateral pelvic nerve stimulation. The interval between stimulations was 10-15 min to prevent nerve exhaustion.

Measurement of Genital Tissue Hemoglobin Concentration.

Tissue hemoglobin concentration was determined by a near infrared optical spectroscopy technique using a dual channel laser oximeter (model 96208; ISS, Inc., Champaign, Ill.). This technique utilizes a continuous wave optical spectrometer with a fiber optic array probe consisting of one detector and 8 light sources. The sources emit light in the near infrared spectrum (700-900 nm) which can penetrate a targeted volume of tissue (˜530 mm³) to an average depth of 7.2 mm beneath the optical fibers. By detecting scattered light, absorption by oxyhemoglobin (OHb) and deoxyhemoglobin can be quantified and total hemoglobin can be calculated. Laser oximetry can thus be utilized to assess local tissue hemodynamics in a non-invasive and continuous fashion. The skin around the labia was carefully shaved to ensure good contact with the optical fibers. The probe (2 cm in length) was positioned longitudinally on the skin overlying the clitoris, labia and vagina, such that the detector fiber was positioned just below the pubic arch. The probe assembly was secured in place by a metal stand. The area over the probe was covered with a black cloth to prevent any interference from natural or artificial ambient light sources.

Drug Administration.

Topical Intravaginal Drug Administration.

A flexible 18 gauge catheter was attached to a syringe loaded with hydrogel formulation. The catheter was filled until the formulation reached 3-5 mm from the open end. The catheter was placed 2.5 cm into the vagina and secured to the labia with suture to prevent migration. The syringe was placed on the heating pad to keep the formulation warm. Following control nerve stimulations, 1 ml of formulation was infused into the vagina. Pelvic nerve stimulation was repeated 15 min and 30 min afterwards. Nerve stimulation at the 15 min time point coincided with the time of maximal serum concentration.

Vaginal Submucosal Injection of Drug.

Rec 15/2615 hydrochloride (1 mg/ml) was dissolved in 10% (v/v) N,N-dimethylformamide. Prior to the application of the laser oximeter probe, Rec 15/2615 hydrochloride solution was loaded into two 23 gauge infusion sets connected to 1 ml syringes (50 μl per infusion set). Needles were inserted into the distal vaginal wall, just below the mucosal surface, at the 3 and 9 o'clock positions. Following control nerve stimulations, drug was infused into the vagina. Pelvic nerve stimulation was repeated 15 min and 30 min after drug administration.

Treatment Groups.

The following animals groups were treated:
Group I. Intravaginal application of 1 ml of vehicle (placebo).
Group II. Intravaginal application of 1 ml of formulation containing 0.075% Rec 15/2615 hydrochloride, described in Example 1, Table 1, Batch 50.
Group III. Intravaginal application of 1 ml of formulation containing 0.15% Rec 15/2615 hydrochloride, described in Example 1, Table 1, Batch 51.
Group IV. Intravaginal application of 1 ml of formulation containing 0.30% Rec 15/2615 hydrochloride, described in Example 1, Table 1, Batch 52.
Group V. Vaginal submucosal injection of 100 μg/100 μl of Rec 15/2615 hydrochloride.

Measurement of Plasma Blood Concentration of Rec 15/2615.

Blood samples (˜2 ml) were obtained from the carotid artery prior to formulation administration and then 14 min after formulation administration. Samples were collected in heparinized syringes and transferred into pre-cooled polypropylene tubes on ice. Plasma was obtained by centrifugation at 1200×g for 2 min at 4° C. and stored at −20° C.

Samples Preparation.

0.1 mL of rabbit plasma was transferred to an Eppendorf tube and 10 μL of internal standard methanolic solution (0.2 μg/mL of 4-amino-6,7-dimethoxy-2-[4-(2-isopropyl-5-methylphenoxy acetyl)-1-piperazinyl]quinazoline (Rec 15/2693) in methanol) were added. 1 mL of acetonitrile was added to each sample and samples were shaken on a multimixer shaker for 1 min. Protein was allowed to precipitate and samples were then centrifuged at 4° C. for 10 min at 2700 g. The organic layer was transferred to a new glass tube and evaporated at 45° C. under a stream of nitrogen. The residue was dissolved in 250 μL of buffer solution containing methanol/10 mM ammonium formate, pH 3.5 (40:60; v/v). Samples were mixed 1 min using a vortex mixer and clarified by centrifugation (5 min×2700 g). The clarified solution was transferred into an autosampler vial. Aliquots of 20 μL were analyzed by LC/MS-MS.

Data Analysis.

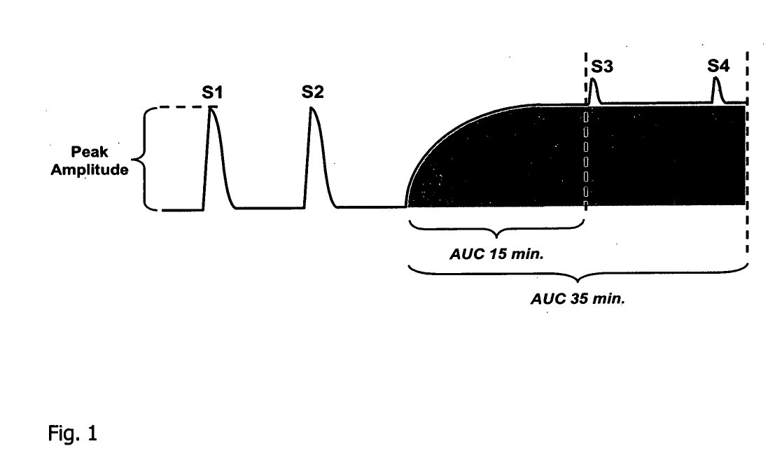

Change in peak amplitude of oxyhemoglobin concentration following nerve stimulation was determined as the difference between the average peak tissue oxyhemoglobin concentration and baseline. For assessment of genital engorgement after formulation application or drug injection, the baseline was subtracted from the tissue oxyhemoglobin recording and the area-under-the-curve (AUC) was determined for the first 15 min, the time between 15-35 min and for the complete the period (0-35 min) following formulation administration or submucosal injection. Increases in oxyhemoglobin following nerve stimulation were not included in the AUC determinations (see FIG. 1). Comparisons between three or more groups were analyzed by one-way ANOVA, followed by Dunnett post-hoc test. Comparisons within a group with matched data were analyzed by paired t-test or repeated measures ANOVA followed by Dunnett post-hoc test. When the variances among groups were not homogenous (Bartlett's test), data were transformed using the equation [logX or RADQ(X+10)].

### RESULTS

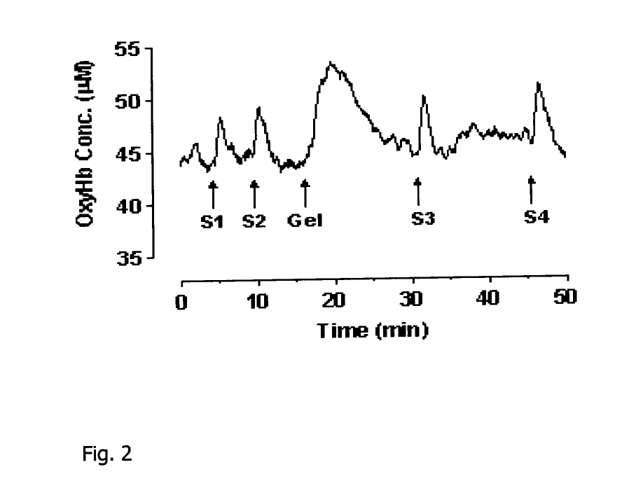

Submaximal pelvic nerve stimulation for 30 sec caused a rapid and transient increase in genital tissue oxyhemoglobin (FIG. 1). Repeated nerve stimulation did not significantly change the characteristics of the oxyhemoglobin peaks. Topical vaginal administration of control formulation containing no active ingredient (vehicle) caused a transient increase in genital tissue OxyHb. The observed increase in OxyHb levels following topical administration was similar in magnitude to the increase observed following pelvic nerve stimulation (FIG. 2). The increase in OxyHb may be a measurement artifact due to the movement of the vaginal wall when formulation is introduced. Displacement of tissues beneath the probe, however, usually results in an instantaneous change in the OxyHb recording. Such an abrupt change was rarely observed during formulation instillation. Furthermore, the transient nature of this response suggests that there may be an actual increase in blood flow due to physical stimulation of the vaginal wall by the viscous formulation.

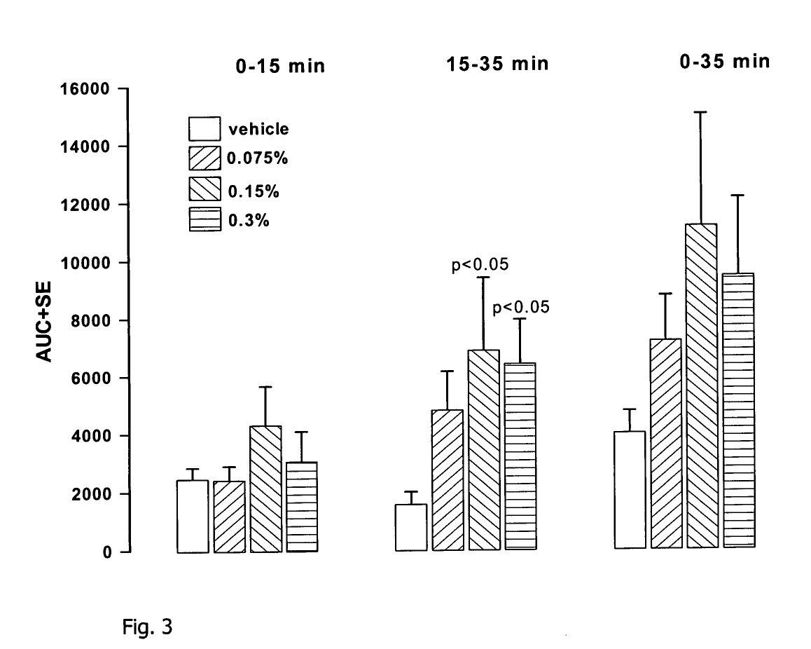

Comparisons of the observed levels of genital engorgement for control formulation without active ingredient and formulations containing Rec 15/2615 showed that treatment with Rec 15/2615 had a sustained effect compared to treatment with control formulation (FIG. 3). Control formulation again showed a transient increase in genital tissue OxyHb observable at 0-15 min after administration. With the exception of the 0.15% concentration, the mean area-under-the-curve (AUC) was similar for Rec 15/2615 and vehicle at 0-15 min. The mean AUC evaluated at 15-35 min revealed a statistically significant difference for the groups treated with 0.15% and 0.3% concentrations, compared to vehicle-treated control group. At 0-35 min, a dose-dependent trend was noted with higher AUC values relative to vehicle. Hence, in contrast to administration of control formulation without active ingredient, administration of formulation containing Rec 15/2615 caused a sustained increase in genital tissue OxyHb. Genital engorgement was consistently sustained throughout the duration of the experimental protocol, up to 45 minutes following formulation administration. Hence, topical treatment with Rec 15/2615 maintained genital engorgement well past the period of transient engorgement observed following administration of control formulation without active ingredient.

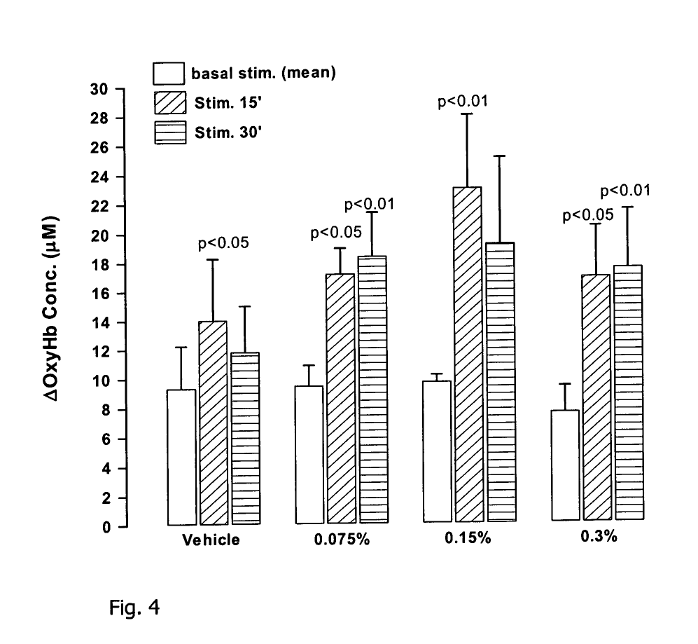

Topical vaginal application of Rec 15/2615 also significantly increased genital tissue OxyHb in response to pelvic nerve stimulation (FIG. 4). Consistent with the observation that treatment with control formulation led to a transient increase in genital engorgement, treatment with control formulation lacking active ingredient exhibited a significant nerve-induced increase in OxyHb at 15 min. The effect of treatment with control treatment, however, was attenuated at 30 min. Treatment with formulation containing Rec 15/2615, by contrast, lead to a significant increase in nerve-induced increase in OxyHb that was observable at 15 min and which remained high at 30 min.

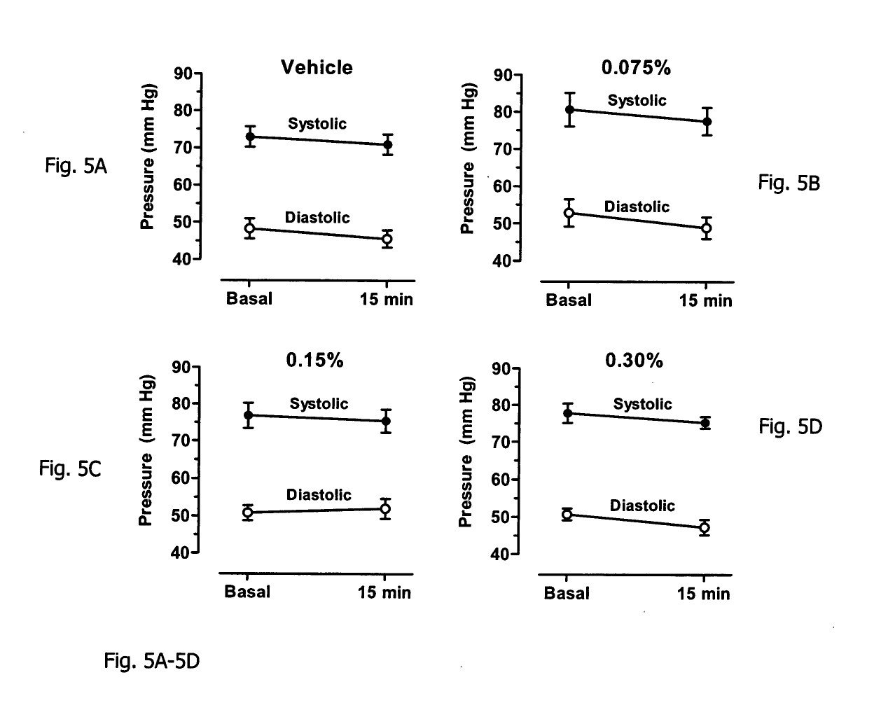

Topical vaginal administration of Rec 15/2615 had no significant effect on blood pressure, indicating that the effect of Rec 15/2615 is exerted preferentially on the vaginal/clitoral vessels (FIG. 5).

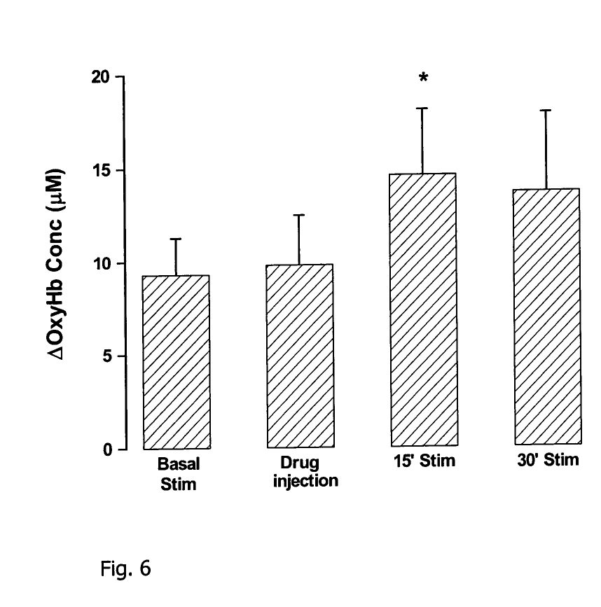

Submucosal injection of Rec 15/2615 produced a rise in genital tissue oxyhemoglobin that was similar to pelvic nerve stimulation (FIG. 6). Pelvic nerve stimulation, 15 min after drug injection, produced a significantly enhanced response. This enhancement appeared to be maintained at 30 min, although the response did not reach statistical significance when compared to control stimulations. Also after submucosal injection, Rec 15/2615 did not affect systemic blood pressure (data not shown). Rec 15/2615 plasma concentrations evaluated 15 min after intravaginal application of 1 ml of formulation containing different concentrations of active molecule, as well as direct submucosal injection of 0.1 mg/animal of the compound are summarized in Table 4.

TABLE 4
Mean plasma levels (ng/ml) of Rec 15/2615 15 min after vaginal
application of 1 ml of formulation at different concentrations, in
comparison with plasma levels obtained after direct submucosal
injection of 0.1 mg of the compound.

| | Formulation: | Formulation: | Formulation: | Submucosal |
|---|---|---|---|---|
| | 0.075% | 0.15% | 0.3% | injection |
| Mean | 10.6 | 45.2 | 72.5 | 26.3 |
| S.D. | 6.7 | 18.7 | 33.5 | 33.8 |

A dose-dependent increase of Rec 2615 levels was obtained increasing the concentration of active compound into the formulation.

### CONCLUSION

These data demonstrate that topical vaginal application of the hydrogel containing Rec 15/2615 hydrochloride rapidly and significantly enhanced genital engorgement without affecting systemic blood pressure. From a comparison of the effects observed following application of formulation containing Rec 15/2615 hydrochloride at the two lower concentrations (0.075% and 0.15%) with the effects observed after direct submucosal injection of the compound (FIGS. 4 and 6), and the plasma levels of the active compound 15 min after the application/injection, it appears that the effect of the compound when administered as a gel is higher than that obtained by direct injection. The data also demonstrate that genital engorgement responses may be prolonged after topical administration of Rec 15/2615 and its salts in the manner of the present invention.

1. A transmucosal delivery preparation comprising a therapeutically effective amount of Rec 15/2615, or a pharmaceutically acceptable salt thereof, and a
(i) solubilizing agent, penetration enhancer, and thickening agent; or
(ii) solubilizing agent, penetration enhancer, and thickening and emulsifying agent,
said preparation adapted to deliver a therapeutic amount of Rec 15/2615 across a female genital mucosal membrane.

2. The transmucosal delivery preparation according to claim 1 which further comprises a humectant.

3. The transmucosal delivery preparation according to claim 1 consisting essentially of a therapeutically effective amount of Rec 15/2615, or a pharmaceutically acceptable salt thereof, and
(i) a solubilizing agent, a penetration enhancer, and a thickening agent; or
(ii) a solubilizing agent, a penetration enhancer, and a thickening and emulsifying agent.

4. The transmucosal delivery preparation according to claim 1 which comprises a cyclodextrin as a solubilizing agent.

5. The transmucosal delivery preparation according to claim 1 wherein said thickening and emulsifying agent is Sepigel™ 305.

6. The transmucosal delivery preparation according to claim 1 which comprises diethyleneglycol monoethyl ether as a solubilizing agent and penetration enhancer and hydroxyethylcellulose as a thickening agent.

7. The transmucosal delivery preparation according to claim 2 which comprises diethyleneglycol monoethyl ether as a solubilizing agent and penetration enhancer, hydroxyethylcellulose as a thickening agent, and propylene glycol as a humectant.

8. The transmucosal delivery preparation according to claim 7 which comprises from about 30% to about 50% diethyleneglycol monoethyl ether, from about 0.5% to about 5% hydroxyethylcellulose, and about 0.1% to about 20% propylene glycol.

9. The transmucosal delivery preparation of claim 6 which comprises from about 30% to about 50% diethyleneglycol monoethyl ether and from about 1% to about 5% hydroxyethylcellulose.

10. The transmucosal delivery preparation of claim 9 which further comprises from about 5% to about 10% propylene glycol, as a humectant.

11. The transmucosal delivery preparation of claim 1 which comprises from about 0.005% to about 2.5% Rec 15/2615.

12. The transmucosal delivery preparation of claim 1 further including an additional active agent.

13. The transmucosal delivery preparation of claim 12 wherein said additional active agent is selected from the group consisting of prostaglandins, direct vasodilators, and type-V phosphodiesterase inhibitors, or two or more of the foregoing.

14. The transmucosal preparation of claim 13 wherein said prostaglandin is prostaglandin E₂.

15. The transmucosal preparation of claim 13 wherein said direct vasodilator is papaverine.

16. The transmucosal preparation of claim 13 wherein said type-V phosphodiesterase inhibitor is selected from the group consisting of 1-{[3-(4,7-dihydro-1-methyl-7-oxo-3-propyl-1H-pyrazolo[3,4-d]pyrimidin-5-yl-4-ethoxyphenyl]-4-methylpiperazine (sildenafil), 2-[2-ethoxy-5-(4-ethylpiperazine-1-ylsulphonyl)phenyl]-7-propylimidazo[5,1-f][1,2,4]triazin-4(3H)-one (vardenafil), and (6R,12aR)-6-(1,3-benzodioxol-5-yl)-2-methyl-1,2,3,4,6,7,12,12a-octahydropyrazino[1′,2′:1,6]pyrido[3,4-b]indole-1,4-dione (tadalafil).

17. A method for treating female sexual dysfunction in a patient in need of such treatment, comprising transmucosally administering the preparation of claim 1 to the genital mucosa of said patient, to treat said female sexual dysfunction.

18. The method of claim 17 wherein said preparation is administered to the vaginal mucosa of said patient.

19. The method of claim 17 comprising topically administering the preparation to a genital mucosal membrane of said patient.

20. The method of claim 19 wherein the composition is administered to the vaginal mucosa of said patient.

21. The method of claim 17 further comprising administering one or more active agent selected from the group consisting of prostaglandins, direct vasodilators, and type-V phosphodiesterase inhibitors.

22. A method for enhancing female sexual responsiveness comprising topically administering to a genital mucosal membrane of a female individual the preparation of claim 1 comprising an amount of Rec 15/2615 or pharmaceutically acceptable salt thereof sufficient to increase the blood flow in the clitoris or vaginal wall of said female individual, thereby enhancing the sexual responsiveness of said female individual.

23. The method of claim 22 herein the composition is administered to the vaginal mucosa of said female individual.

24. The method of claim 22 further comprising administering one or more active agent selected from the group consisting of prostaglandins, direct vasodilators, and type-V phosphodiesterase inhibitors.

25. A method of administering Rec 15/2615 or a pharmaceutically acceptable salt thereof transmucosally to a female patient, which comprises contacting a genital mucous membrane of said patient with a composition according to claim 1, and maintaining contact of said composition with said genital mucous membrane for a sufficient time period to deliver a therapeutically effective amount of the Rec 15/2615 or pharmaceutically acceptable salt thereof to said patient.

26. The method of claim 25 further comprising administering one or more active agent selected from the group consisting of prostaglandins, direct vasodilators, and type-V phosphodiesterase inhibitors.
